(19) United States
(12) Patent Application Publication
(10) Pub. No.: US 2024/0023792 A1
(43) Pub. Date: Jan. 25, 2024

(54) MODIFIED BALL-TIP ARCHITECTURE FOR MEDICAL OPTICAL FIBER

(71) Applicant: LUMENIS LTD., Yokneam (IL)

(72) Inventors: Asaf Granot, Lotem (IL); Arkady Khachaturov, Haifa (IL); Alex Travisky, Karmiel (IL); Haim Bar-Natan, Shlomy (IL)

(73) Assignee: LUMENIS LTD., Yokneam (IL)

(21) Appl. No.: 18/356,514

(22) Filed: Jul. 21, 2023

Related U.S. Application Data
(60) Provisional application No. 63/391,172, filed on Jul. 21, 2022.

(51) Int. Cl. A61B 1/00

(52) U.S. Cl. CPC A61B 1/0017

(57) ABSTRACT

The present disclosure provides a modified ball-tip architecture for a medical optical fiber where the modified ball-tip provides both protection to the distal end of the optical fiber and a working channel of an endoscope in which the medical optical fiber is inserted as well as provides for pre-procedure measurements. The modified ball-tip provides a ball-tip but exposes the distal end of the optical fiber.

### CROSS-REFERENCE TO RELATED APPLICATIONS

This application claims the benefit of priority under 35 U.S.C. § 119 of U.S. Provisional Application No. 63/391,172, filed Jul. 21, 2022, the entire disclosure of which is hereby incorporated by reference.

### TECHNICAL FIELD

The present disclosure generally relates to medical optical fibers. Particularly, but not exclusively, the present disclosure relates to protective ball tips on the distal end of the medical optical fiber.

### BACKGROUND

Medical optical fibers typically include an optical fiber and a jacket surrounding the optical fiber. Optical fibers may comprise an innermost optical core and optionally a cladding layer surrounding the optical core. Some optical fibers can optionally include a mechanical support layer surrounding the cladding layer. Additionally, medical optical fibers often include jackets (or protective layers), and many have portions of their jackets stripped at a distal end, for example, like electrical wires, which leaves a stripped optical fiber bare optical fiber distal section. Medical optical fiber diameters are in the range of a few tens of microns to a few hundreds of microns. Due to this small diameter, the distal section of the medical optical fiber can be sharp or pointed.

Some medical optical fibers include a smooth tip disposed on a distal end for assisting in passage through an endoscope or body lumen. Such tip additions are designed to shatter, fragment, melt, or otherwise be destroyed, when laser pulses are activated to expose the optical fiber core. Some tips provide mechanical strength to the medical optical fiber.

### BRIEF SUMMARY

The present disclosure provides a medical optical fiber comprising an optical fiber disposed along a longitudinal axis. The medical optical fiber further includes an optical fiber tip disposed on a distal end of the medical optical fiber where the distal end of the medical optical fiber comprises an optical fiber tip having an optical fiber end face transverse to the longitudinal axis. Additionally, the optical fiber includes an innermost optical core having an optical core end face central to the optical fiber end face. The medical optical fiber further includes a jacket surrounding the optical fiber where the jacket has a distal jacket face end transverse to the longitudinal axis and the jacket comprises at least one aperture.

Further provided herein is a modified ball-tip, or protective tip, encircling an exposed portion of the optical fiber in a region where the jacket has been stripped. The modified ball-tip radially encircles an inner layer of the medical optical fiber proximate to the optical fiber end face.

With some examples, a medical optical fiber comprises an optical fiber jacket, an inner optical fiber layer disposed within the optical fiber jacket, a length of the inner optical fiber layer exposed at a distal end of the medical optical fiber, the inner optical fiber layer comprising at least a fiber core, a modified ball-tip radially encircling the exposed inner optical fiber layer proximate to a distal end of the inner optical fiber layer, at least a portion of a distal end of the fiber core exposed.

In some examples, the inner optical fiber layer comprising a cladding layer, the fiber core disposed within the cladding layer, the modified ball-tip disposed radially around the cladding layer. In some examples, a distal front edge of the modified ball-tip is less than 10 micrometers away from the distal end of the fiber core. In some examples, a distal front edge of the modified ball-tip is between 0 and 50 micrometers away from the distal end of the fiber core longitudinally towards the proximal end of the medical optical fiber. In some examples, the distal front edge of the modified ball-tip comprising a flattened shape. In some examples, the distal frond edge of the modified ball-tip comprising a rounded shape. In some examples, wherein the exposed length of the inner optical fiber layer is between 2.5 and 3.5 millimeters (mm). In some examples, wherein the modified ball-tip extends along the exposed length of the inner optical fiber layer longitudinally a distance between 400 and 1500 micrometers (μm). In some examples, wherein the modified ball-tip extends along the exposed length of the inner optical fiber layer longitudinally a distance between 450 and 550 micrometers (μm). In some examples, wherein a diameter of the modified ball-tip is between 500 and 550 micrometers (μm).

With some examples, a method of manufacturing a medical optical fiber, comprises providing a medical optical fiber, the medical optical fiber comprising an optical fiber jacket and an inner optical fiber layer disposed within the optical fiber jacket, the inner optical fiber layer comprising at least a fiber core, stripping a portion of the optical fiber jacket to expose a length of the inner optical fiber layer at a distal end of the medical optical fiber, applying an adhesive to a portion of the exposed length of the inner optical fiber layer proximate to the distal end of the medical optical fiber to form a modified ball-tip, at least a portion of the distal end of the fiber core exposed through the modified ball-tip.

In some examples, the adhesive is Vitralit® 6108T glue. In some examples, the method comprises polishing the modified ball-tip to expose the distal end of the fiber core. In some examples, the method comprises placing the portion of the exposed length of the inner optical fiber layer in a mold and injecting the adhesive into the mold to form the modified ball-tip. Some examples provide a medical optical fiber formed using the above example methods.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a conventional medical optical fiber with a ball-tip.

FIG. 2 illustrates a medical optical fiber with a modified ball-tip, in accordance with examples of the disclosure.

FIG. 3 illustrates an alternative view of the medical optical fiber of FIG. 2.

FIG. 4 illustrates another alternative view of the medical optical fiber of FIG. 2.

FIG. 5 illustrates an exploded view of a portion of the medical optical fiber of FIG. 2.

FIG. 6 illustrates a method of manufacture in accordance with at least one embodiment.

FIG. 7 illustrates another medical optical fiber with a modified ball-tip, in accordance with examples of the disclosure.

FIG. 8 illustrates an alternative view of the medical optical fiber of FIG. 7.

FIG. 9 illustrates another alternative view of the medical optical fiber of FIG. 7.

FIG. 10 illustrates an exploded view of a portion of the medical optical fiber of FIG. 7.

### DETAILED DESCRIPTION

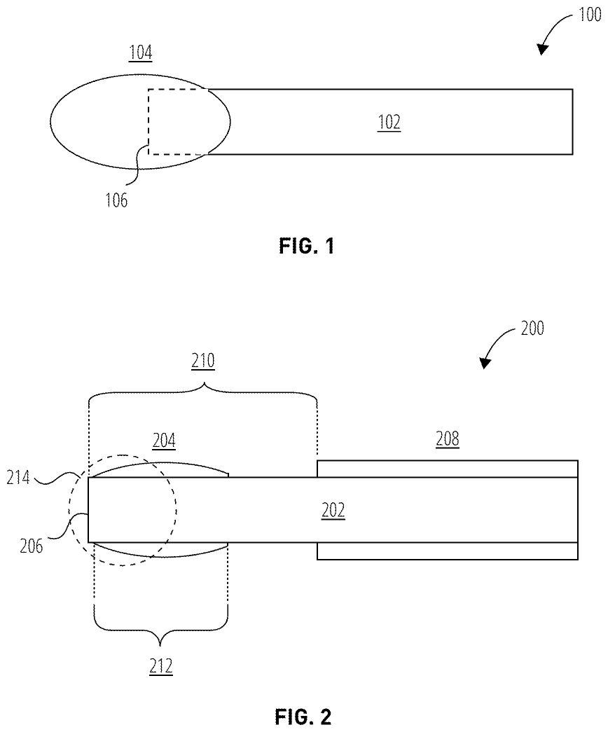

As noted, conventional optical fibers can include a ball-tip. FIG. 1 illustrates a medical optical fiber 100. Medical optical fiber 100 includes a fiber 102 with a ball-tip 104 disposed over, or enclosing, the distal end 106 of the fiber 102. Such conventional ball-tip designs have several disadvantages. For example, ball-tip 104 does not allow for automatic calibration procedures or advanced diagnostics features. That is, ball-tip 104 interferes with optical beams emitted prior to a laser beam having enough energy to destroy the ball-tip 104.

Additionally, empirical evidence tends to show that ball-tips (e.g., ball-tip 104) may provide protection to the distal end 106 of the fiber 102 from destruction or degradation during treatment. However, since conventional ball-tip 104 are blown off or destroyed immediately (e.g., at the beginning of a procedure) this protection is lost due to the design of the conventional ball-tip 104.

The present disclosure provides a modified ball-tip architecture that advantageously allows for pre-procedure (e.g., pre therapeutic laser beam activation) measurements and diagnostics using optical beams as well as provides protection to the distal end of the fiber once the procedure starts while still protecting the working channel of an endoscope of body lumen. FIG. 2 illustrates medical optical fiber 200, according to some embodiments of the present disclosure. Medical optical fiber 200 includes a fiber 202, which can include an optical fiber core, a cladding layer, a mechanical support layer, or the like. The medical optical fiber 200 further includes a modified ball-tip 204 disposed at the distal end 206 of fiber 202. Further, this figure depicts fiber jacket 208. As can be seen, the fiber jacket 208 is stripped a length 210 from the distal end 206.

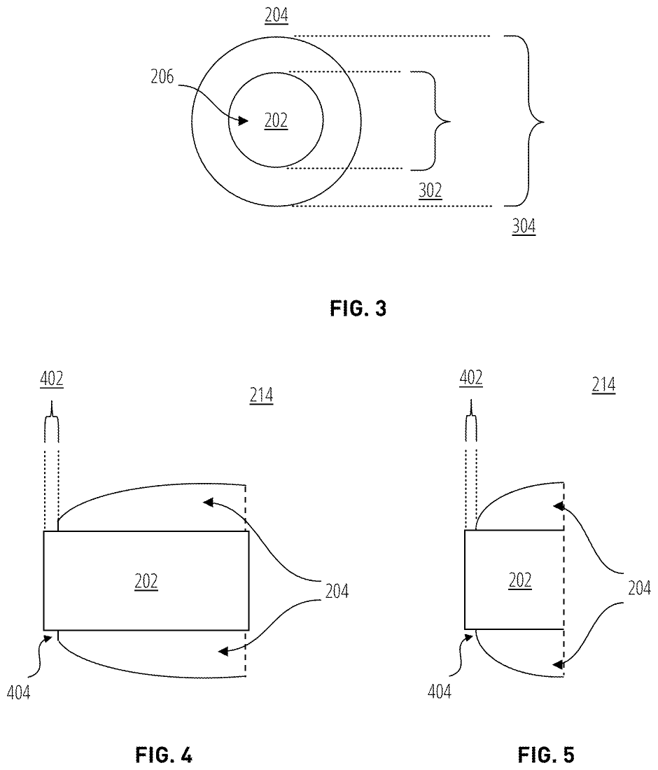

Modified ball-tip 204 extends along the exposed length of fiber 202 (e.g., longitudinally) length 212. As can be seen, modified ball-tip 204 does not completely enclose the distal end 206 of fiber 202 and does not extend back to the fiber jacket 208. This is further illustrated in FIG. 3, which shows a view from the distal end 206 of fiber 202.

As depicted in FIG. 3, the modified ball-tip 204 encircles (e.g., radially) the fiber 202. However, the distal end 206 of fiber 202 is exposed. Further, diameter 302 of fiber 202 as well as diameter 304 of modified ball-tip 204 are depicted.

Additionally, with the modified ball-tip 204 architecture depicted in FIG. 2 and illustrated with medical optical fiber 200, the modified ball-tip 204 may not extend to the distal end 206 of fiber 202. For example, FIG. 4 illustrates exploded view 214 from FIG. 2. As can be seen in exploded view 214 shown in FIG. 4, the modified ball-tip 204 extends along a longitudinal length of fiber 202 to within distance 402 of distal end 206. As illustrated in this figure, the modified ball-tip 204 may have a flattened distal front edge 404. In other examples, the distal front edge 404 may be rounded.

For example, FIG. 5 illustrates exploded view 214 showing modified ball-tip 204 having distal front edge 404 rounded (e.g., as opposed to flattened as shown in FIG. 3). With some examples, length 210 can be between 2 and 4 millimeters (mm). In alternative examples, length 210 can be between 2.5 and 3.5 mm. In some examples, length 212 can be between 400 and 1500 micrometers (μm). With alternative examples, length 212 can be between 500 and 600 μm or between 550 and 750 μm. With some examples, diameter 302 of fiber 202 can be between 50 and 500 μm. With alternative embodiments, diameter 302 of fiber 202 can be between 150 and 350 μm.

With some examples, diameter 304 of modified ball-tip 204 can be between 400 and 500 μm. In alternative embodiments, diameter 304 of modified ball-tip 204 can be between 450 and 500 μm or between 500 and 550 μm. In some examples, distance 402 from distal end 206 to distal front edge 404 (e.g., front edge, edge closest to distal end 206, or the like) of modified ball-tip 204 can be between 0 and 50 μm. In alternative examples, distal front edge 404 is less than 10 μm (e.g., between 0 and 10 μm, less than 10 μm, or the like) from distal end 206.

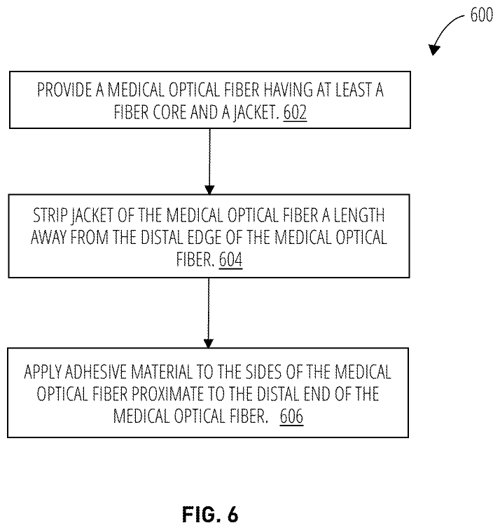

FIG. 6 illustrates a method 600 of manufacturing a medical optical fiber. The method 600 can be implemented to manufacture medical optical fibers like the medical optical fiber 200 shown herein. Method 600 can begin at block 602. At block 602 “provide a medical optical fiber” a medical optical fiber can be provided. With some examples, the provided medical optical fiber can have fiber 202 (e.g., including a fiber optic core, a cladding layer, and optionally additional layers) surrounded or enclosed by a fiber jacket 208.

Continuing to block 604 “strip jacket of the medical optical fiber a length away from the distal edge of the medial optical fiber” the fiber jacket 208 is stripped length 210 away from distal end 206. Exposing a portion of the medical optical fiber. For example, fiber jacket 208 can be stripped exposing the mechanical support layer, the cladding layer, the fiber optic core, or another layer disposed beneath the fiber jacket 208. In some examples, method 600 can exclude block 604. Said differently, with some examples, the modified ball-tip 204 can be formed over the fiber jacket 208. As such, block 604 can be omitted, fiber jacket 208 will not be stripped from the inner fiber layer, and the modified ball-tip 204 can be formed over the fiber jacket such that a distal face at the distal end 206 of the fiber 202 is exposed as described herein.

Continuing to block 606 “apply adhesive material to the sides of the medical optical fiber proximate to the distal end of the medical optical fiber” adhesive material (e.g., a bio-compatible adhesive, Vitralit® 6108T glue, or the like) is applied to the exposed portion of the medical optical fiber 200 (e.g., in the region where fiber jacket 208 is stripped and proximate to the distal end 206. In some examples, modified ball-tip 204 can be formed from a non-adhesive material. For example, modified ball-tip 204 can be formed from a bio-compatible material with sufficient mechanical adhesion (with or without adhesive material) to stay on the distal end 206 of the medical optical fiber 200 during manufacturing, while packaged, during insertion into a working channel of an endoscope, and/or during use in a procedure.

With some examples, the adhesive is applied (e.g., at block 606) using a dispenser and rolling apparatus to form modified ball-tip 204 as depicted above. With some embodiments, method 600 includes a block to cure or harden the applied adhesive material. In some embodiments, the adhesive is applied (e.g., at block 606) using a mold to form a shape (e.g., having flatted distal front edge 404, or the like). With some embodiments, block 606 applies adhesive to form a conventional ball-tip (e.g., ball-tip 104) and method 600 includes a further block (not shown) to polish the ball-tip 104 until the fiber 202 is exposed revealing distal end 206.

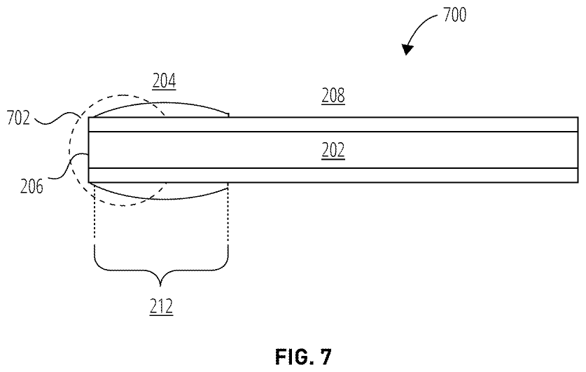

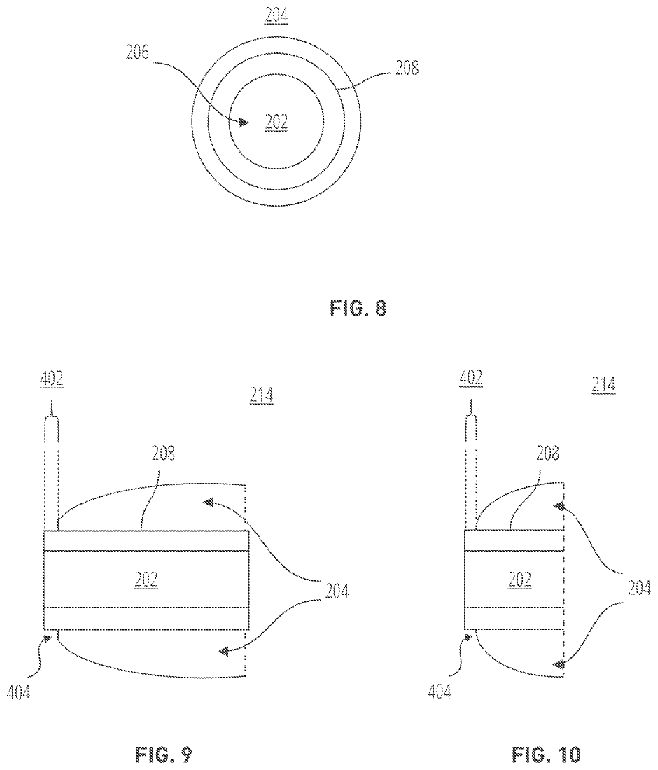

FIG. 7 illustrates a medical optical fiber 700, according to some embodiments of the present disclosure. Medical optical fiber 700 includes a fiber 202, which can include an optical fiber core, a cladding layer, a mechanical support layer, or the like. The medical optical fiber 700 further includes a modified ball-tip 204 disposed at the distal end 206 of fiber 202. Further, this figure depicts fiber jacket 208. However, unlike the medical optical fiber 200 shown in FIG. 2, the fiber jacket 208 is not stripped from the distal end 206. As such, modified ball-tip 204 extends along the fiber jacket 208 (e.g., longitudinally) length 212. As can be seen, modified ball-tip 204 does not completely enclose the most distal end face of the fiber 202 at distal end 206. This is further illustrated in FIG. 8, which shows a view of medical optical fiber 700 from the distal end 206 of fiber 202.

As depicted in FIG. 8, the modified ball-tip 204 encircles (e.g., radially) the fiber jacket 208, which itself encircles (e.g., radially) the fiber 202. However, the distal end 206 of fiber 202 is exposed.

Additionally, with the modified ball-tip 204 architecture depicted in FIG. 7 and illustrated with medical optical fiber 700, the modified ball-tip 204 may not extend to the distal end 206 of fiber 202. For example, FIG. 9 illustrates exploded view 702 from FIG. 7. As can be seen in exploded view 702 shown in FIG. 9, the modified ball-tip 204 extends along a longitudinal length of fiber 202 to within distance 402 of distal end 206. As illustrated in this figure, the modified ball-tip 204 may have a flattened distal front edge 404. In other examples, the distal front edge 404 may be rounded.

For example, FIG. 10 illustrates exploded view 214 showing modified ball-tip 204 having distal front edge 404 rounded (e.g., as opposed to flattened as shown in FIG. 8).

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is the inclusive “or” rather than the exclusive or, and indicates at least one of, or any combination of items it conjoins.

While the presented concepts have been described with respect to a limited number of embodiments, it will be appreciated that many variations, modifications, and other applications of the disclosure can be implemented without departing from the scope of the appended claims.

What is claimed is:

1. A medical optical fiber, comprising:
an optical fiber jacket;
an inner optical fiber layer disposed within the optical fiber jacket, a length of the inner optical fiber layer exposed at a distal end of the medical optical fiber, the inner optical fiber layer comprising at least a fiber core;
a modified ball-tip radially encircling the exposed inner optical fiber layer proximate to a distal end of the inner optical fiber layer, at least a portion of a distal end of the fiber core exposed.

2. The medical optical fiber of claim 1, the inner optical fiber layer comprising a cladding layer, the fiber core disposed within the cladding layer, the modified ball-tip disposed radially around the cladding layer.

3. The medical optical fiber of claim 1, wherein a distal front edge of the modified ball-tip is less than 10 micrometers away from the distal end of the fiber core.

4. The medical optical fiber of claim 1, wherein a distal front edge of the modified ball-tip is between 0 and 50 micrometers away from the distal end of the fiber core longitudinally towards the proximal end of the medical optical fiber.

5. The medical optical fiber of claim 4, the distal front edge of the modified ball-tip comprising a flattened shape.

6. The medical optical fiber of claim 4, the distal frond edge of the modified ball-tip comprising a rounded shape.

7. The medical optical fiber of claim 1, wherein the exposed length of the inner optical fiber layer is between 2.5 and 3.5 millimeters (mm).

8. The medical optical fiber of claim 1, wherein the modified ball-tip extends along the exposed length of the inner optical fiber layer longitudinally a distance between 400 and 1500 micrometers (μm).

9. The medical optical fiber of claim 1, wherein the modified ball-tip extends along the exposed length of the inner optical fiber layer longitudinally a distance between 450 and 550 micrometers (μm).

10. The medical optical fiber of claim 1, wherein a diameter of the modified ball-tip is between 500 and 550 micrometers (μm).

11. A method of manufacturing a medical optical fiber, comprising:
providing a medical optical fiber, the medical optical fiber comprising an optical fiber jacket and an inner optical fiber layer disposed within the optical fiber jacket, the inner optical fiber layer comprising at least a fiber core;
stripping a portion of the optical fiber jacket to expose a length of the inner optical fiber layer at a distal end of the medical optical fiber; and
applying an adhesive to a portion of the exposed length of the inner optical fiber layer proximate to the distal end of the medical optical fiber to form a modified ball-tip, at least a portion of the distal end of the fiber core exposed through the modified ball-tip.

12. The method of claim 11, wherein the adhesive is Vitralit® 6108T glue.

13. The method of claim 11, comprising polishing the modified ball-tip to expose the distal end of the fiber core.

14. The method of claim 11, comprising placing the portion of the exposed length of the inner optical fiber layer in a mold and injecting the adhesive into the mold to form the modified ball-tip.

15. A medical optical fiber, comprising:
an optical fiber jacket;
an inner optical fiber layer disposed within the optical fiber jacket, a length of the inner optical fiber layer exposed at a distal end of the medical optical fiber, the inner optical fiber layer comprising at least a fiber core;
a modified ball-tip radially encircling the exposed inner optical fiber layer proximate to a distal end of the inner optical fiber layer, at least a portion of a distal end of the fiber core exposed, a distal front edge of the modified ball-tip comprising a flattened shape.

16. The medical optical fiber of claim 15, the inner optical fiber layer comprising a cladding layer, the fiber core disposed within the cladding layer, the modified ball-tip disposed radially around the cladding layer.

17. The medical optical fiber of claim 15, wherein a distal front edge of the modified ball-tip is less than 10 micrometers away from the distal end of the fiber core.

18. The medical optical fiber of claim 15, wherein a distal front edge of the modified ball-tip is between 0 and 50 micrometers away from the distal end of the fiber core longitudinally towards the proximal end of the medical optical fiber.

19. The medical optical fiber of claim 15, wherein the exposed length of the inner optical fiber layer is between 2.5 and 3.5 millimeters (mm).

20. The medical optical fiber of claim 15, wherein the modified ball-tip extends along the exposed length of the inner optical fiber layer longitudinally a distance between 400 and 1500 micrometers (nm).
